(19) United States
(12) Patent Application Publication
(10) Pub. No.: US 2006/0127473 A1
(43) Pub. Date: Jun. 15, 2006

(54) Compositions and methods for stabilizing active pharmaceutical ingredients

(76) Inventor: William Michael Nichols, Somerset, NJ (US)

Correspondence Address: Barry H. Jacobsen;Legal Division, Warner-Lambert Company, LLC, 201 Tabor Road, Morris Plains, NJ, 07950, US

(21) Appl. No.: 11/301,609

(22) Filed: Dec. 13, 2005

Related U.S. Application Data
(60) Provisional application No. 60/635,450, filed on Dec. 13, 2004.

(51) Int. Cl. A61K 31/717; A61K 31/137; A61K 9/20

(52) U.S. Cl. 424/464; 514/57; 514/649

(57) ABSTRACT

The present invention relates to stable pharmaceutical compositions having an unstable active pharmaceutical ingredient.

This application claims priority to US provisional patent application No. 60/635,450, filed Dec. 13, 2004.

### FIELD OF THE INVENTION

The present invention relates to stable pharmaceutical compositions having an unstable active pharmaceutical ingredient.

### DESCRIPTION OF RELATED ART

It is important for active pharmaceutical ingredients (APIs) to be stable for prolonged periods of time. Generally, at least a two year shelf life is desirable. Unfortunately, APIs often may not be stable alone or in combination with excipients that are used in dosage formulations such as tablets, capsules, films, etc.

Many API's may be unstable and degrade in the presence of oxygen. Oxygen sensitive API's may be stabilized by several different methods. For instance, the drug product and the API may be processed under inert atmospheres, e.g. under argon or nitrogen gas blankets, however, this requires special manufacturing conditions and adds to the cost of manufacturing a drug product. Antioxidants may be used to help stabilize oxygen sensitive API's. Useful antioxidants include bi-sulfites and ascorbic acid, however, there have been safety issues associated with the use of some antioxidants. In particular, sulfites have been determined to be harmful. Thus, the use of antioxidants is less desirable due to safety issues and it generally costs more than preparing a nasal spray solution under inert conditions.

Accordingly, it would be desirable to provide stable compositions containing unstable API's, such as oxygen sensitive API's, and methods of providing such stable compositions.

### SUMMARY

Several embodiments of the present invention provide for a pharmaceutical composition including at least one active pharmaceutical ingredient in a pharmaceutically effective amount and silicified microcrystalline cellulose in a stabilizing effective amount. The composition may be stable where the active ingredient(s) experience minimal or no degradation, e.g. less than 10% degradation when stored at room temperature over a two year period of time. The composition may be a tablet including a direct compressed tablet.

The at least one active pharmaceutical ingredient may be phenylephrine, a derivative or a pharmaceutically acceptable salt thereof, in a pharmaceutically effective amount. Phenylephrine may be present in an amount from about 5 to about 12 milligrams. In several embodiments, the phenylephrine does not experience more than 10% degradation when stored at room temperature over a two year period of time. In several embodiments, a tablet may be an immediate release orally digested tablet, an immediate release bucally fast disintegrating tablet or a sustained release orally ingested tablet. In several embodiments, there is provided a package including the composition. The package may include drug facts. The composition may be positioned in blister containers.

Several embodiments provide for a composition including at least one active pharmaceutical ingredient in a pharmaceutically effective amount. The active pharmaceutical ingredient may be phenylephrine, a derivative or a pharmaceutically acceptable salt thereof. The composition may be a direct compressed tablet. The composition may include silicon dioxide and microcrystalline cellulose or silicified microcrystalline cellulose.

Further embodiments of the present invention provide for a method of stabilizing at least one active pharmaceutical ingredient including the step of combining silicified microcrystalline cellulose in a stabilizing effective amount to at least one active pharmaceutical ingredient present in a pharmaceutically effective amount. The active pharmaceutical ingredient may be unstable in the presence of oxygen. Other embodiments of the present invention provide for a method of using silicified microcrystalline cellulose to stabilize at least one unstable active pharmaceutical ingredient including the step of combining the silicified microcrystalline cellulose in a stabilizing effective amount with at least one active pharmaceutical ingredient present in a pharmaceutically effective amount. An active pharmaceutical ingredient may be oxygen sensitive and include phenylephrine.

Further embodiments of the present invention provide for a method of manufacturing a stable phenylephrine tablet including the step of mixing a dry blend of said phenylephrine in a pharmaceutically effective amount with silicified microcrystalline cellulose and other pharmaceutically acceptable excipients. The method may further include the step of compressing the mixture to form a directly compressed tablet.

### DETAILED DESCRIPTION

Silicified microcrystalline cellulose (SMCC) and microcrystalline cellulose are known excipients that may be used in pharmaceutical formulations as a binder and/or as a means to increase the processability of the formulation. SMCC and microcrystalline cellulose are frequently used in combination with each other or interchangeably. However, this invention has discovered a distinct and surprising difference between SMCC and microcrystalline cellulose. It has been surprisingly found that silicified microcrystalline cellulose can stabilize unstable active pharmaceutical ingredients (API's). Other typical binders, including microcrystalline cellulose, reducing sugars, such as lactose, dicalcium phosphate, and the like do not stabilize such APIs and/or provide other undesirable characteristics, including content uniformity or processability issues.

Silicified MCC is a particulate agglomerate of co-processed microcrystalline cellulose and from about 0.1% to about 20% silicon dioxide, by weight of the microcrystalline cellulose, the microcrystalline cellulose and silicon dioxide being in intimate association with each other, and the silicon dioxide portion of the agglomerate being derived from a silicon dioxide having a particle size from about 1 nanometer (nm) to about 100 microns (μm), based on average primary particle size. By “intimate association”, it is meant that the silicon dioxide has in some manner been integrated with the microcrystalline cellulose particles, e.g., via a partial coating of the microcrystalline particles, as opposed to a chemical interaction of the two ingredients. The term “intimate association” is therefore deemed for purposes of the present description as being synonymous with “integrated” or “united”. The coprocessed particles are not necessarily uniform or homogeneous. Rather, under magnification, e.g., scanning electron microscope at 500 times, the silicon dioxide at the preferred percent inclusion appears to be an “edge-coating”. Preferably, the silicon dioxide comprises from about 0.5% to about 10% of the silicified MCC, and most preferably from about 1.25% to about 5% by weight relative to the microcrystalline cellulose. Moreover, the silicon dioxide preferably has a particle size from about 5 nm to about 40 μm, and most preferably from about 5 nm to about 50 μm. Moreover, the silicon dioxide preferably has a surface area from about 10 m 2 g to about 500 m2/g, preferably from about 50 m 2/g to about 500 m2/g, and more preferably from about 175 m2/g to about 350 m2/g. Silicified MCC, and methods for its manufacture, are described in U.S. Pat. No. 5,585,115, the entire disclosure of which is incorporated herein by reference. Silificified microcrystalline cellulose is commercially available from Penwest Pharmaceuticals, Inc., under the trademark Prosolv®. Prosolv is available in a number of grades, including, for example, Prosolv SMCC 50, Prosolv SMCC 90, and Prosolv HD, each of which contains 2% colloidal silicon dioxide, by weight relative to the microcrystalline cellulose.

SMCC may be present in a composition in an amount from about 0.1% to about 99.5%, from about 25 to about 95% or from about 50 to about 85% by weight of the total formulation.

An embodiment of the present invention provides for a stable formulation having an API and silificied microcrystalline cellulose (SMCC). An embodiment of the present invention provides for a stable formulation having an unstable API in a pharmaceutically effective amount and silificied microcrystalline cellulose (SMCC) in a stabilizing effective amount. SMCC is desirably in an amount that is sufficient to stabilize the API. An API may be unstable for a variety or reasons including, but not limited, to sensitivities, e.g. degradation is caused by exposure to moisture, heat, oxygen or other compounds in a composition. Another embodiment provides a stable formulation having an oxygen sensitive API and silicified microcrystalline cellulose. An oxygen sensitive API is a pharmaceutically active compound that is unstable in the presence of oxygen.

The term ‘unstable’ refers to APIs that degrade more than 10% when stored at room temperature over a two year period of time and ‘stable’ refers to APIs that do not degrade more than 10% when stored at room temperature over a two year period of time. The degradation of an API compound could be physical, chemical, structural or combinations thereof.

In several of the embodiments of the present invention the stability of an oxygen sensitive API is increased and/or enhanced by combining or mixing the API with silicified microcrystalline cellulose (SMCC).

Some examples of oxygen-sensitive materials which are subject to degradation due to oxygen exposure include, but is not limited to, amines either as salts or as free bases, sulfides, allylic alcohols, phenols and the like. Often oxygen sensitive APIs may have pKa values in the range from about 1 to about 10, more particularly in the range from about 5 to about 9. Also oxygen sensitive API's may have redox potentials less than or equal to about 1300 mV versus Ag/Ag⁺, or less than or equal to about 1000 mV versus Ag/Ag⁺. Examples of some specific oxygen sensitive API's include, but are not limited to, phenylephrine, pseudoephedrine, tiagabine, acitretin, rescinnamine, lovastatin, tretinoin, isotretinoin, simvastatin, ivermectin, verapamil, oxybutynin, hydroxyurea, selegiline, esterified estrogens, tranylcypromine, carbamazepine, ticlopidine, methyidopahydro, chlorothiazide, methyidopa, naproxen, acetominophen, erythromycin, bupropion, rifapentine, penicillamine, mexiletine, verapamil, diltiazem, ibuprofen, cyclosporine, saquinavir, morphine, sertraline, cetirizine, N-[[2-methoxy-5-(1-methyl) phenyl]methyl]-2-(diphenylmethyl)-1-azabicylco[2.2.2]octan-3-amine and the like.

In various embodiments of the present invention, the oxygen sensitive API is phenylephrine. Phenylephrine is known to experience physical and chemical degradation. Degradation of phenylephrine may be caused by a variety of factors including, but not limited to, the presence of oxygen, moisture, reducible sugars, bases, high temperatures, etc. Degradation of phenylephrine may be noticed by a change in color, e.g. changing from a whitish color to a darker, blackish color. Additionally, phenylephrine may degrade chemically as recorded by degradation peaks during analysis, such as an HPLC analysis. It is desirable to prevent, reduce or minimize the degradation of phenylephrine. An embodiment of the present invention provides a stable pharmaceutical composition having phenylephrine in a therapeutically effective amount and silicified microcrystalline cellulose in an amount sufficient to stabilize phenylephrine.

Phenylephrine may be used as a nasal decongestant and is an example of an oxygen sensitive API. There are several nasal spray formulations for the treatment of nasal decongestion that contain phenylephrine. Marketed nasal sprays containing phenylephrine may be stabilized by manufacturing the spray under inert atmospheres, e.g. under argon or nitrogen gas blankets, which requires special manufacturing conditions and is costly. Interestingly, there are no currently marketed solid dosage forms containing phenylephrine. One reason for this may be due to the instability of phenylephrine in the presence of oxygen. Surprisingly, it has been found that SMCC increases the stability of phenylephrine in solid formulations, whereas microcrystalline cellulose does not enhance the stability of an oxygen sensitive API. Thus, SMCC advantageously may be used simultaneously for its known properties such as a binder and a processing aide and a stabilizer for phenylephrine.

Useful amounts of phenylephrine include from about 1 milligram to about 60 mg, or from about 1 mg to about 15 mg, or from about 5 mg to about 10 mg or from about 5 mg to about 10 mg.

Various embodiments of the present invention provide compositions with at least two API's. An additional embodiment provides for a composition with three API's, of which at least one API is an oxygen sensitive API. Useful additional API's include, but are not limited to:
(a) antimicrobial agents such as triclosan, cetylpyridium chloride, domiphen bromide, quaternary ammonium salts, zinc compounds, sanguinarine, fluorides, alexidine, octonidine, EDTA, and the like;
(b) non-steroidal anti-inflammatory and pain reducing agents such as aspirin, acetaminophen, ibuprofen, ketoprofen, diflunisal, fenoprofen calcium, flurbiprofen sodium, naproxen, tolmetin sodium, indomethacin, celecoxib, valdecoxib, parecoxib, rofecoxib and the like;
   (c) antitussives such as benzonatate, caramiphen edisylate, menthol, dextromethorphan hydrobromide, chlophedianol hydrochloride and the like;
   (d) antihistamines such as brompheniramine maleate, chlorpheniramine maleate, carbinoxamine maleate, clemastine fumarate, dexchlorpheniramine maleate, diphenylhydramine hydrochloride, azatadine maleate, diphenhydramine citrate, diphenhydramine hydrochloride, diphenylpyraline hydrochloride, doxylamine succinate, promethazine hydrochloride, pyrilamine maleate, tripelennamine citrate, triprolidine hydrochloride, acrivastine, loratadine, desloratadine, brompheniramine, dexbropheniramine, fexofenadine, cetirizine, montelukast sodium and the like;
(e) expectorants such as guaifenesin, ipecac, potassium iodide, terpin hydrate and the like;
(f) analgesic-antipyretics such salicylates, phenylbutazone, indomethacin, phenacetin and the like;
(g) antimigraine drugs such as sumitriptan succinate, zolmitriptan, valproic acid eletriptan hydrobromide and the like.

The amount of the additional API's in the formulation may be adjusted to deliver a predetermined dose of the active agent over a predetermined period of time, which may typically vary from 4 to 24 hours. Examples of doses containing specific pharmaceutically active agents are set forth in Table 1.

TABLE 1

| Pharmaceutically Active Agent | Dose | |
|---|---|---|
| Chlorpheniramine Maleate | 4-12 | mg |
| Brompheniramine Maleate | 4 | mg |
| Dexchlorpheniramine | 2 | mg |
| Dexbropheniramine | 2 | mg |
| Triprolidine Hydrochloride | 2.5 | mg |
| Cetirizine | 5-10 | mg |
| Acrivastine | 8 | mg |
| Azatadine Maleate | 1 | mg |
| Loratadine | 5-10 | mg |
| Dextromethorphan Hydrobromide | 10-30 | mg |
| Ketoprofen | 12.5-25 | mg |
| Sumatriptan Succinate | 35-70 | mg |
| Zolmitriptan | 2.5 | mg |
| Nicotine | 1-15 | mg |
| Diphenhydramine Hydrochloride | 12.5-25 | mg |
| Atorvastatin | 5-80 | mg |
| Valdecoxib | 5-20 | mg |
| Celecoxib | 5-20 | mg |
| Rofecoxib | 5-25 | mg |
| Ziprasidone | 20-80 | mg |
| Eletriptan | 10-40 | mg |

Except as otherwise noted, the amount of API is designated as % by weight per dosage form. Generally, the amount of the API used may be from about 0.01% to about 80% by weight, or from about 0.1% to about 40% by weight, or from about 1% to about 30% by weight, or from about 1% to about 10% by weight.

An “effective” amount or a “therapeutically effective amount” of an active ingredient refers to a non-toxic but sufficient amount of the agent to provide the desired effect. The amount of active agent that is “effective” will vary from subject to subject, depending on the age and general condition of the individual, the particular active agent or agents, and the like. Thus, it is not always possible to specify an exact “effective amount.” However, an appropriate “effective” amount in any individual case can be determined by one of ordinary skill in the art using routine experimentation.

“Pharmacologically active” (or simply “active”), refers to a compound that has pharmacological activity and a “pharmacologically active” derivative of an active agent, refers to a derivative having the same type of pharmacological activity as the parent compound and approximately equal in degree. When the term “pharmaceutically acceptable” is used to refer to a derivative (e.g., a salt) of an active agent, it is to be understood that the compound is pharmacologically active as well. When the term “pharmaceutically acceptable” is used to refer to an excipient, it implies that the excipient has met the required standards of toxicological and manufacturing testing or that it is on the Inactive Ingredient Guide prepared by the Food and Drug Administration.

By “pharmaceutically acceptable” such as in the recitation of a “pharmaceutically acceptable excipient,” or a “pharmaceutically acceptable additive,” is meant a material that is not biologically or otherwise undesirable, i.e., the material can be incorporated into a pharmaceutical composition administered to a patient without causing any undesirable biological effects or interacting in a deleterious manner with any of the other components of the composition in which it is contained.

In various embodiments of the present invention, the dosage forms may be administered orally. Oral administration may involve swallowing, so that the composition with the API(s) enters the gastrointestinal tract, and/or buccal, lingual, or sublingual administration by which the API enters the blood stream directly from the mouth.

Formulations suitable for oral administration include solid, semi-solid and liquid systems such as tablets, soft or hard capsules containing multi- or nano-particulates, liquids, or powders; lozenges (including liquid-filled); chewable tablets; fast disintegrating or fast dissolving tablets; gels; fast dispersing dosage forms; films; ovules; granules, wafers, gums, capsules, caplets, powders, sprays, and buccal/mucoadhesive patches. In one embodiment, a fast disintegrating dosage form is contemplated where a dry mixture of the components of the invention gives rise, upon direct compression, to fast disintegrating tablets having a disintegration time of less than about 60 seconds or less than about 30 seconds or less than about 15 seconds in the oral cavity.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

Useful inactive ingredients, include but are limited to, binding agents, filling agents, lubricating agents, suspending agents, sweeteners, flavorings and flavor enhancer agents, taste-masking agents, preservatives, buffers, wetting agents, anti-oxidants, colorants or coloring agents, pharmaceutically acceptable carriers, disintegrants, salivary stimulating agents, cooling agents, co-solvents (including oils), pH adjusting agents, effervescent agents, emollients, bulking agents, anti-foaming agents, surfactants, soluble organic salts, permeabilizing agents, glidants and other excipients and combinations thereof. Desirably, the agents are chemically and physically compatible with the API.

Useful pH adjusting agents include fumaric acid, citric acid, sodium acetate. Useful surfactants include sorbitan esters, docusate sodium, sodium lauryl sulfate, cetriride. Useful soluble organic salts include sodium carbonate, sodium bicarbonate, sodium chloride. Examples of useful binding agents include, but are not limited to, polyethylene glycols, soluble hydroxyalkylcelluloses, polyvinylpyrrolidone, gelatins, natural gums, various celluloses and cross-linked polyvinylpyrrolidone, microcrystalline cellulose, such as Avicel® PH101 and Avicel® PH102.

Examples of useful substantially water soluble carriers or filling agents include, but are not limited to, various starches, celluloses, carbohydrates compression sugars or soluble fillers. More particularly, useful fillers include but are not limited to lactose, lactose monohydrate, lactose anhydrous, sucrose, amylose, dextrose, mannitol, inositol, maltose, maltitol, sorbitol, glucose, xylitol, erythritol, fructose, maltodextrins; microcrystalline cellulose, calcium carboxy methyl cellulose; pregelatinized starch, modified starches, potato starch, maize starch; clays, including kaolin and polyethylene glycols (PEG) including PEG 4000; or combinations thereof. Useful amount of fillers include the range of about 1 to about 99 weight percent, or about 25 to about 95 weight percent or about 40 weight percent to about 95 weight percent of the compositions of this invention.

Compositions of the present invention may include a sweetener. Useful sweeteners include, but are not limited to, sugars such as sucrose, glucose (corn syrup), dextrose, invert sugar, fructose, and mixtures thereof; acid saccharin and its various salts such as the sodium or calcium salt; cyclamic acid and its various salts such as the sodium salt; the dipeptide sweeteners such as aspartame and alitame; natural sweeteners such as dihydrochalcone compounds; glycyrrhizin; Stevia rebaudiana (Stevioside); sugar alcohols such as sorbitol, sorbitol syrup, mannitol, xylitol and the like, synthetic sweeteners such as acesulfame-K and sodium and calcium salts thereof and other synthetic sweeteners, hydrogenated starch hydrolysate (lycasin); protein based sweetening agents such as talin (thaumaoccous danielli) and/or any other pharmacologically acceptable sweetener known by the state of the art, and mixtures thereof.

Suitable sugar alcohols useful as sweeteners include, but are not limited to, sorbitol, xylitol, mannitol, galactitol, maltitol, isomalt (PALATINIT™) and mixtures thereof. The exact amount of sugar alcohol employed is a matter of preference subject to such factors as the degree of cooling effect desired. Thus, the amount of sugar alcohol may be varied in order to obtain the result desired in the final product and such variations are within the capabilities of those skilled in the art without the need for undue experimentation.

In another embodiment, the formulations according of the invention are free of sugar. A sugar-free formulation has the advantage that it can be administered easily to consumers with blood sugar disorders or to diabetics in need of such preparations. Such sweeteners include, but are not limited to, sucralose, acesulfame potassium, and aspartame which share properties such as absence of bitter and metallic aftertastes.

In another embodiment, a composition may include acesulfame K, aspartame, sucralose and combinations thereof. Acesulfame K is a commercial product of Nutrinova Nutrition Specialties & Food Ingredient GmbH. Useful amounts of sucralose in a dosage form is between about 0.002% to about 10% by total weight of the FDDF. However, this amount can vary greatly depending upon the nature of the composition being sweetened. In one preferred embodiment, the sweetener is a mixture of sucralose with acesulfame K. The tablets may be uncoated, however, they can, if desired, be coated with any suitable coating agent known in the art. Suitable coating agents are those used for immediate release purposes and will disintegrate in saliva. Such coatings include, but are not limited to, hydroxypropyl methylcellulose, or methyl cellulose, or OPADRY™ and the like and combinations thereof.

One embodiment of the invention provides for a controlled or extended release composition having SMCC and a controlled release of therapeutically active pharmaceutical ingredients, such as phenylephrine.

Optionally, one or more flavors such as those described in U.S. Pat. No. 6,596,298 which is incorporated herein. Any amount of flavor can be used and will depend on characteristics of the active pharmaceutical ingredient(s); preferred concentration of flavoring is between about 0.01% to about 10% w/w of a composition.

A tablet disintegrant may be added to the direct compression process for its wicking (i.e., the ability of particles to draw water into the porous network of a tablet) and swelling ability. Some disintegrants also serve as excellent binders and are able to substantially improve the mechanical strength of the formulation. Suitable disintegrants are carboxymethyl cellulose sodium, carboxymethyl cellulose calcium, crospovidone, sodium starch glycolate, corn starch, insoluble cationic-exchange resins such as polyacrylin, microcrystalline cellulose, croscarmellose. Disintegrants can be added at a concentration ranging from about 0.5% to about 30%. Croscarmellose sodium (cross-linked carboxymethyl cellulose) may be present at a concentration of about 2% to about 10%.

An effective amount of any generally accepted pharmaceutical tableting lubricant can be added to compress the tablets. An amount within the range from about 0.25% to about 6%, or 0.5% to about 3% by weight can be added. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

One or more glidant materials which improve the flow of the powder blend and minimize the dosage form weight variation can be used. Useful glidants include but are not limited to silicone dioxide, talc and combinations thereof.

The invention can further provide a taste-masked oral pharmaceutical composition including coating or encapsulating the systemically active therapeutic agent with a suitable coating material. Examples of suitable coating materials for taste-masking include polymers such as hydroxypropylmethylcellulose, ethylcellulose, methacrylates, methacrylate co-polymers such as Eudragit® (Butylmethacrylat-(2-Dimethylaminoethyl)methacrylat-Methylmethacrylat-Copolymer (1:2:1)”), KOLLICOAT®, and polyvinylpyrrolidone. The pharmaceutical composition can include other functional components presented for the purpose of modifying the physical, chemical or taste properties of the systemically active therapeutic agent. For example, the systemically active therapeutic agent can be in the form of microencapsulation, ion-exchange resin complex, such as a sulfonated polymers, electro-chemical melt, supercritical fluids, magnesium trisilicate, coacervation, or cyclodextrin (cyclic-linked oligosaccharides) complexes. Useful sulphonated polymers include polystyrene cross-linked with 8% of divinylbenzene such as Amberlite ®IRP-69 and IRP-64 (obtained by Rohm and Haas), Dow XYS-40010.00®, Dow XYS40013.00® (obtained from the Dow Chemical Company).

The dose, pKa and solubility of the drug molecule influences formulation and taste masking methods. It is understood that any method in the art for masking the taste of pharmaceuticals to facilitate their oral administration can be used. For example, taste masking can also be achieved by simple wet granulation or roller compaction with other excipients to minimize presented surface area of the drug. Spray drying can also be used to taste mask the systemically active therapeutic agent.

It is further contemplated that the pharmaceutically active ingredients can be added in the form of an encapsulate. Encapsulation can be achieved using conventional procedures and can be performed using water-insoluble as well as water-soluble agents. Alternatively, it is possible to encapsulate a release controlling substance, together with the systemically active therapeutic agent, within an encapsulating shell to provide for controlled release of the taste-masked oral pharmaceutical composition.

An embodiment of the present invention provides for a process for preparing a tablet formulation. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. Freeze or spray drying may also be used. The formulation of tablets is discussed in Pharmaceutical Dosage Forms: Tablets, Vol. 1, by H. Lieberman and L. Lachman (Marcel Dekker, New York, 1980). Direct compression is a relatively quick process where the powdered materials are compressed directly without changing the physical and chemical properties of the drug. Direct compression excipients are chosen such that they have good flow and compressible characteristics and prevent segregation of powders in the hopper and thereby help in direct compression. For example, tablets may be obtained by blending together the active drug ingredient(s) and SMCC, and optional inactive ingredients, and optionally other therapeutically active ingredients and excipients to form a homogeneous mixture; blending together; and directly compressing the mixture.

In another embodiment, the dosage form composition is a film prepared by any suitable method for producing fast dissolving film such as those described in U.S. Pat. No. 6,596,298 issued to Leung et al, which is incorporated herein. Consumable oral films for human or veterinary use are typically pliable water-soluble or water-swellable thin film dosage forms which may be rapidly dissolving or mucoadhesive and typically include an API, a film-forming polymer, a binder, a solvent, a humectant, a plasticiser, a stabiliser or emulsifier, a viscosity-modifying agent and a solvent. Some components of the formulation may perform more than one function. Films may be manufactured by conventional processes such as those disclosed in U.S. Pat. Nos. 3,784,390; 4,927,636; 6,177,096, each of which is incorporated herein. The film-forming polymer may be selected from natural polysaccharides, proteins, or synthetic hydrocolloids and is typically present in the range 0.01 to 99 weight %, more typically in the range 30 to 80 weight %. Useful water soluble film forming polymers are described in U.S. Pat. No. 6,596,298 to Leung et al. and include, but are not limited to, polyvinyl alcohol, pullulan, hydroxypropylmethylcellulose, hydroxyethylcellulose, hydroxypropylcellulose, polyvinylpyrrolidone, carboxymethyl cellulose, polyvinyl alcohol, sodium alginate, polyethylene glycol, xanthan gum, tragacanth gum, guar gum, acacia gum, arabic gum, polyacrylic acid, methylmethacrylate copolymer, carboxyvinyl polymer, amylose, high amylose starch, hydroxypropylated high amylose starch, dextrin, pectin, chitin, chitosan, levan, elsinan, collagen, gelatin, zein, gluten, soy protein isolate, whey protein isolate, casein and mixtures thereof. A particularly useful water soluble polymer is pullulan. The dry film can be cut to suitable size and shape for unit dose pouching.

Solid formulations for oral administration may be formulated to be immediate and/or modified controlled release. Controlled release formulations include modified release formulations include delayed-, sustained-, pulsed-, controlled-, targeted and programmed release.

Suitable modified release formulations for the purposes of the invention are described in US Pat. No. 6,106,864, which is incorporated herein. Details of other suitable release technologies such as high energy dispersions and osmotic and coated particles are to be found in Pharmaceutical Technology On-line, 25(2), 1-14, by Verma et al (2001), which is incorporated herein. The use of chewing gum to achieve controlled release is described in WO 00/35298, which is incorporated herein.

Another embodiment of the present invention provides a kit having two or more separate compositions having an API and SMCC and a means for separately retaining said compositions, such as a container, divided bottle, or divided foil packet. An example of such a kit is the familiar blister pack used for the packaging of tablets, capsules and the like. Other embodiments contemplate articles of manufacture including various packaging configurations, ranging from unit dose blister packs to multiple dose packages such as bottles. To assist compliance, the kit may have directions for administration and may be provided with a so-called memory aid.

In one embodiment, tablets are advantageously provided in blister packaging which is believed to limit the amount of oxygen that may interact with the composition containing the oxygen sensitive API and as such may also increase or enhance the stability of the drug product containing the oxygen sensitive API. Another embodiment contemplates a method of dispensing a composition from a blister pack by forcing the drug product through a foil back on a blister pack.

### EXAMPLES

TABLE 2

| | Weight (mg/Tablet) | | | | | |
|---|---|---|---|---|---|---|
| | Example # | | | | | |
| Ingredient | 1 | 2 | 3 | 4 | 5 | 6 |
| Phenylephrine HCl | 5.00 | 5.00 | 5.00 | 5.00 | 5.00 | 10.00 |
| Acetaminophen | | 325.00 | 325.00 | 325.00 | 325.00 | |
| Diphenhydramine HCl | | 25.00 | 12.50 | | | |
| Dextromethorphan HBr | | | | 20.00 | | |
| Guaifenesin | | | | | | 200.00 |
| Silicified Microcrystalline | 85.6 | 39.89 | 52.39 | 52.39 | 64.89 | 184.89 |
| Cellulose(Prosolv 90) | | | | | | |
| Starch, pregelatinized, corn | 2.00 | 37.11 | 37.11 | 37.11 | 37.11 | 37.11 |
| Crospovidone USP/EP | 4.00 | 9.00 | 9.00 | 9.00 | 9.00 | 9.00 |
| Stearic acid NF Vegetable Source | 3.00 | 9.00 | 9.00 | 9.00 | 9.00 | 9.00 |
| Magnesium Stearate | 0.40 | | | | | |
| Acesulfame Potassium Salt | 0.152 | | | | | |
| Core weight | 100.152 | 450.00 | 450.00 | 457.50 | 450.00 | 450.00 |
| Blue Film Coating | | 13.50 | | | 13.50 | |
| Green Film Coating | | | 13.50 | | | |
| Yellow Film Coating | | | | 13.50 | | |
| White Film Coating | 6.08 | | | | | 13.50 |
| Wax, Candelilla | | 0.30 | 0.30 | 0.30 | 0.30 | 0.30 |
| Total weight | 106.232 | 463.80 | 463.80 | | 463.80 | 463.80 |

Tablets with the formulation in Table 2 are prepared. Phenylephrine is mixed with silicified microcrystalline cellulose in a drum mixer and then transferred to a tote bin. The additional API's are added to the tote-bin. De-lumped crospovidone, starch and stearic acid is added to the tote bin and blended. De-lumped stearic acid and/or magnesium stearate is added to the tote bin and blended. The blended powder is compressed into a tablet by using a suitable tablet press. The coating material is dispersed in purified water and mixed. The cores as referenced to in Table 2 are loaded into a film coater and continuously sprayed with the coating. Acesulfame potassium salt in purified water is dissolved and Opadry II is dispersed and mixed to form a uniform and de-aerated suspension. This coating suspension is sprayed onto the tablets to attain a smooth and uniform film coating and a target weight of 4% of the white coated tablet weight.

The resulting tablets exhibit acceptable physical characteristics, such as look, color, hardness, etc. The resulting tablets have acceptable stability profiles.

While the invention has been described in detail and with reference to specific examples thereof, it will be apparent to one skilled in the art that various changes and modifications can be made therein without departing from the spirit and scope thereof of the invention.

What is claimed is:

1. A pharmaceutical composition comprising at least one active pharmaceutical ingredient in a pharmaceutically effective amount and silicified microcrystalline cellulose in a stabilizing effective amount.

2. The composition of claim 1, wherein said silicified microcrystalline cellulose is present in an amount from about 0.1% to about 99.5% by weight of said composition.

3. The composition of claim 1, wherein said silicified microcrystalline cellulose is present in an amount from about 25% to about 95% by weight of said composition.

4. The composition of claim 1, wherein said silicified microcrystalline cellulose is present in an amount from about 50% to about 85% by weight of said composition.

5. The compositions of claim 1, wherein said at least one active pharmaceutical ingredient comprises phenylephrine.

6. The composition of claim 5, wherein said phenylephrine is present in an amount from about 1 to about 60 milligrams.

7. The composition of claim 5, wherein said phenylephrine is present from about 5 to about 12 milligrams.

8. The composition of claim 1, wherein said at least one active pharmaceutical ingredient is selected from the group consisting of antimicrobial agents, non-steroidal anti-inflammatory agents, pain reducing agents, decongestants, antitussives, antihistamines, expectorants, analgesic, antipyretics, antipyretics and combinations thereof.

9. The composition of claim 1, wherein said at least one active pharmaceutical ingredient is selected from the group consisting of acetaminophen, phenylephrine, dextromethorphan, guaifenesin, acetaminophen, diphenhydramine, ibuprofen, celecoxib, valdecoxib, chlorpheniramine, triprolidine, cetirizine, sumatriptan, zolmitriptan, loratadine, eletriptan, ketoprofen and combinations thereof.

10. The composition of claim 1, wherein said at least one active pharmaceutical ingredient is selected from the group consisting of acetaminophen, phenylephrine, dextromethorphan, guaifenesin, acetaminophen, diphenhydramine, ibuprofen, cetirizine and combinations thereof.

11. A pharmaceutical composition comprising phenylephrine, derivative or a pharmaceutically acceptable salt thereof, in a pharmaceutically effective amount and silicified microcrystalline cellulose, wherein said pharmaceutical composition is a direct compressed tablet.

12. The composition of claim 11, further comprising at least one additional pharmaceutical ingredient is selected from the group consisting of acetaminophen, phenylephrine, dextromethorphan, guaifenesin, acetaminophen, diphenhydramine, ibuprofen, cetirizine and combinations thereof.

13. The composition of claim 11, wherein said phenylephrine is present in an amount from about 1 to about 60 milligrams.

14. The composition of claim 11, wherein said phenylephrine is present in an amount from about 5 to about 12 milligrams.

15. The composition of claim 11, wherein said formulation is a solid dosage formulation selected from the group consisting of a tablet, capsule, caplet, film, a wafer, granules, powder, a gum, or a chewable tablet which disintegrates with saliva in the mouth.

16. A tablet composition comprising at least one active pharmaceutical ingredient in a pharmaceutically effective amount; wherein said at least one active ingredient comprises phenylephrine, a derivative or a pharmaceutically acceptable salt thereof and said composition is a direct compressed tablet.

17. The composition of claim 16, wherein said composition further comprises silicon dioxide and microcrystalline cellulose.

18. The composition of claim 16, wherein said composition further comprises silicified microcrystalline cellulose.

19. The composition of claim 16, wherein said phenylephrine is present in an amount from about 5 to about 12 milligrams.

20. The composition of claim 16, wherein said at least one active pharmaceutical ingredient is selected from the group consisting of acetaminophen, phenylephrine, dextromethorphan, guaifenesin, acetaminophen, diphenhydramine, ibuprofen, celecoxib, valdecoxib, chlorpheniramine, triprolidine, cetirizine, sumatriptan, zolmitriptan, loratadine, eletriptan, ketoprofen and combinations thereof.
